(19) United States
(12) Patent Application Publication
(10) Pub. No.: US 2012/0180798 A1
(43) Pub. Date: Jul. 19, 2012

(54) ORAL APPLIANCE FOR SLEEP AIRWAY MANAGEMENT

(76) Inventor: Dena BEGG, Spring Lake (CA)

(21) Appl. No.: 13/007,245

(22) Filed: Jan. 14, 2011

(51) Int. Cl. A61F 5/56

(52) U.S. Cl. 128/848

(57) ABSTRACT

An oral appliance for managing a person's airway, includes a malleable ring portion which fits between a user's lower teeth and lower lip, or a user's upper teeth and upper lip; and a tongue depressant portion, which engages the base of a user's tongue to push the tongue downwards and forwards

### FIELD OF THE INVENTION

The present invention is directed to an oral appliance therapeutic device for sleep airway management.

### BACKGROUND

Oral appliances that are used to alleviate snoring and obstructive sleep apnea are devices worn in the mouth, similar to orthodontic retainers or sports mouthguards. They have several advantages over other forms of therapy. Oral appliances are relatively inexpensive, easy to travel with, non-invasive, easy to fabricate, and quite well accepted by patients.

Oral appliances have a definite role in the management of people with airway issues during sleep, such as those with sleep apnea or snoring. The appliance works non-invasively to assist in keeping the airway open. Regardless of how long it is worn, the symptoms of obstructive sleep apnea or snoring will return when the appliance use is discontinued, unless other factors change, such as the user's weight or other physiological trait. There are no guarantees that an appliance will be successful in every patient due to physiologic and anatomic variations and patient tolerance of the appliance.

Oral appliance therapy is becoming recognized by the medical community as an effective treatment option in the management of sleep breathing disorders. Medical and dental researchers have joined together to evaluate and improve the outcome of the use of oral appliances. It is generally recommended that oral appliances be used in patients with primary snoring or mild obstructive sleep apnea and in patients with moderate to severe obstructive sleep apnea who are intolerant of or refuse treatment with nasal continuous positive airway pressure.

Oral appliances fall into two basic categories: tongue-retaining devices (TRD) and mandibular advancing devices (MAD). Tongue-retaining devices are small acrylic devices which fit by suction on the tip of the tongue. Mandibular advancing devices are small acrylic devices that fit over the upper and lower teeth and advance the lower jaw. Both move the base of the tongue forward and open the airway. In general, TRDs do not work as well as MADs. They are non-invasive, easy to use and are effective in improving breathing, reducing snoring and reducing mild sleep apnea. They are less effective for treating people with moderate or severe obstructive sleep apnea and people who are obese.

TRD's are typically custom-made appliances with an anterior bulb that holds the tongue in a forward position during sleep by means of negative pressure (or suction). Currently, the TRD appears to be the appliance of choice for patients who have few or no teeth and for patients who have large tongues. In addition, the TRD is a good appliance for patients who cannot adequately advance their lower jaw for whatever reason. Its disadvantages are that it is more difficult for both clinicians and patients to use on a regular basis, it poses problems for patients who cannot breathe through their nose, it irritates the end of the tongue over time and it is a single jaw and tongue position appliance.

MAD devices fit over both the upper and lower teeth and move the lower jaw forward, which brings the tongue and some throat tissues along with it, opening the airway. They also stimulate the pharyngeal muscles to maintain an open airway during REM sleep—the muscles that normally relax during REM sleep. There are several different designs for MADs. Some designs are not adjustable after fabrication and have to be remade if any alterations are required. Others have an excellent range of adjustment. MAD appliances, in general, require a healthy jaw joint and several healthy teeth with healthy gums in both the upper and lower jaw.

Some of the common side effects with the use of oral appliances are excessive salivation, discomfort in the teeth, dry mouth, tissue irritation from mouth breathing, temporary disharmonies in the bite and some pain in the joint. These side effects are usually short-lived. Upon occasion a poorly fitting crown or filling may be dislodged.

There is a need in the art for an oral appliance which mitigates the disadvantages in the prior art.

### SUMMARY OF THE INVENTION

In one aspect, the invention comprises an oral appliance which comprises:
(a) a malleable ring portion, adapted to fit between a user's lower teeth and lower lip, or a user's upper teeth and upper lip; and
(b) a tongue depressant portion, adapted to engage the base of a user's tongue.

In one embodiment, the appliance comprises a closed loop. In one embodiment, all or a portion of the appliance is fluorescent. The tongue depressant portion may comprise a bulbous enlargement.

In another aspect, the invention comprises a method of preventing or reducing snoring, comprising the step of maintaining pressure on the base of a user's tongue during sleep, with an oral appliance. In one embodiment, the method comprises the step of temporarily placing in a user's mouth, an oral appliance which comprises a ring portion which molds to fit around the user's lower teeth and a tongue depressant portion which engages the base of the user's tongue.

### BRIEF DESCRIPTION OF THE DRAWINGS

In the drawings, like elements are assigned like reference numerals. The drawings are not necessarily to scale, with the emphasis instead placed upon the principles of the present invention. Additionally, each of the embodiments depicted are but one of a number of possible arrangements utilizing the fundamental concepts of the present invention. The drawings are briefly described as follows:

FIG. 1 shows a schematic representation of one embodiment of the oral appliance in position in a user's mouth.

FIG. 2 shows another representation of the oral appliance in position relative to the lower mandible of a user.

FIG. 3 shows one embodiment of the oral appliance.

FIG. 4 shown an alternative embodiment of the ring portion.

FIG. 5 shows yet another alternative embodiment of the ring portion, showing adjustable sizing.

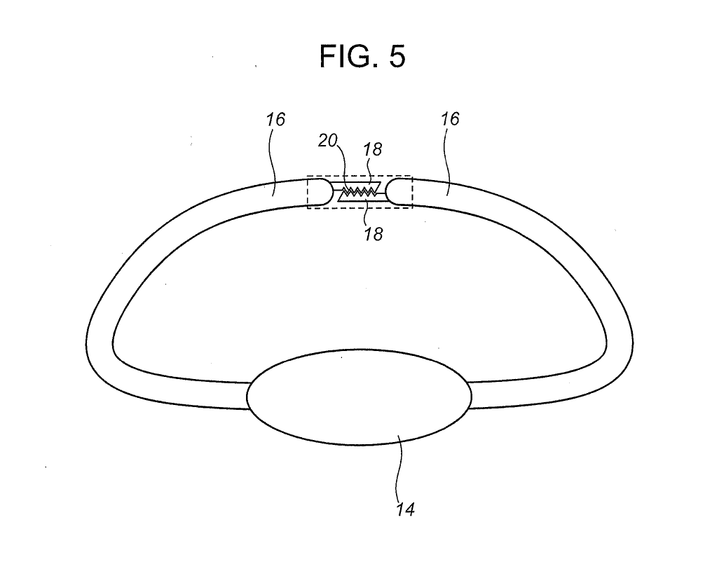

### DETAILED DESCRIPTION OF PREFERRED EMBODIMENTS

The invention relates to an oral appliance for managing a user's airway during sleep. When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

As explained above, conventional airway management oral appliances attach to the tip of a user's tongue, or engage both upper and lower teeth to thrush the lower jaw forward. The present invention provides an alternative which may have advantages over the prior art.

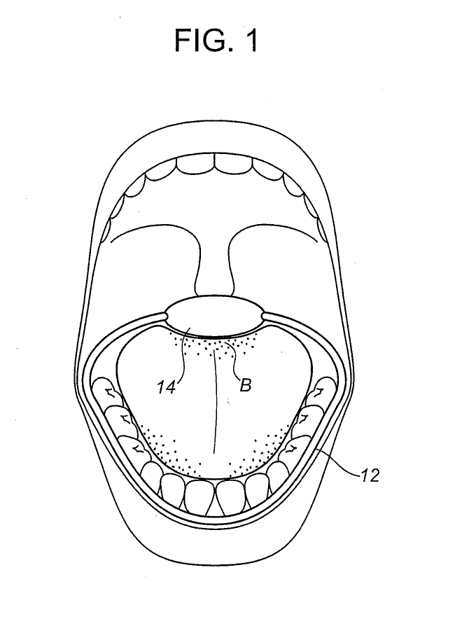

In one embodiment, the appliance comprises a ring portion (12) and a tongue depressant portion (14). The ring portion (12) is relatively thin, such that it can fit comfortably between a user's teeth and lip, as shown in FIG. 1. The ring portion is malleable, such that it is may be easily custom fit to the user's mouth. Preferably, it is not elastically flexible such that it retains its original shape after molding to a particular user. It is preferred that the ring portion retain its new shape after being bent into a desired shape. The ring portion may fit either in the lower lip, or the upper lip.

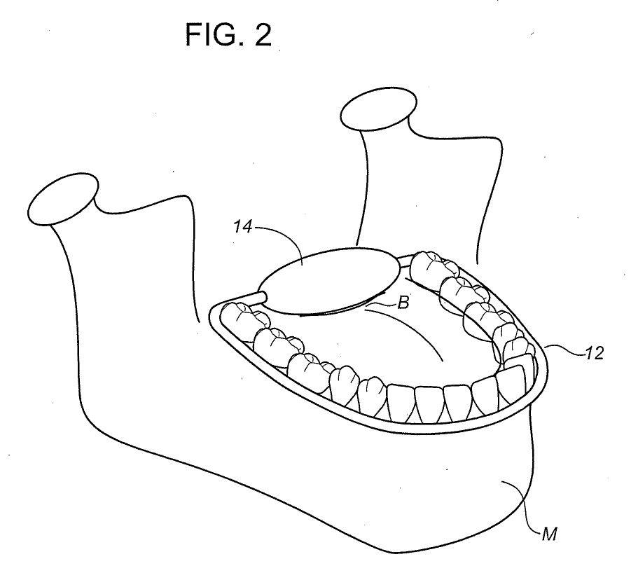

As shown in FIG. 2, in one embodiment, the ring portion (12) is positioned adjacent the lower mandible (M).

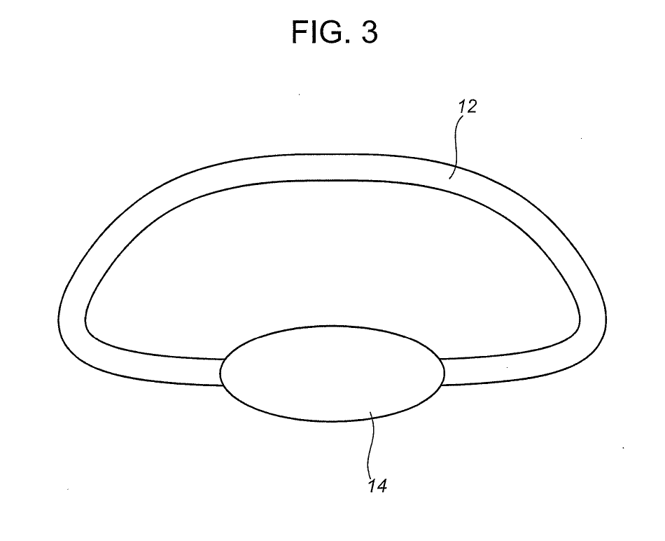

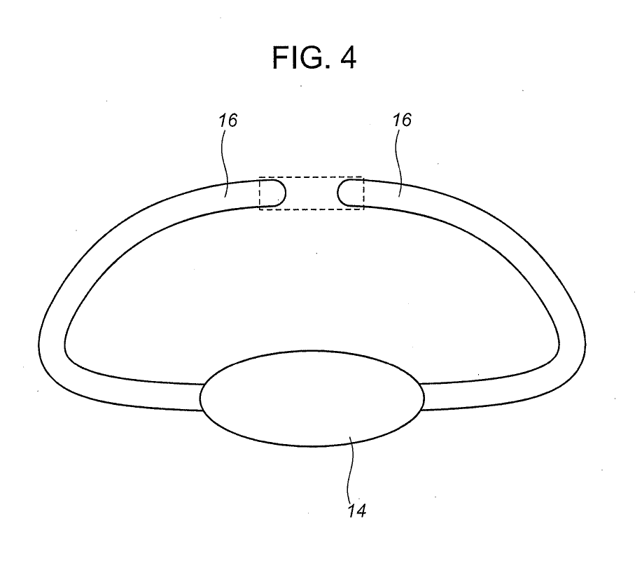

In one embodiment, the ring portion (12) forms a complete, closed ring, as is shown in FIG. 3. In alternative embodiments, the ring portion may have a gap, such that it comprises two extended prongs (16) which do not meet, or are not connected at their distal ends, as shown in FIG. 4.

In one embodiment, the ring portion (12) may be formed by a metal wire encased in soft-touch material such as rubber, silicone rubber, elastomer or a plastic material such as a polyurethane. The metal wire provides the flexible moldability, which allows the ring portion (12) to retain its shape after being molded to a particular shape. The dimensions of the wire and sheath are not essential, except that the ring portion should comfortably fit within a user's lip.

The ring portion may be made in different sizes to accommodate differences in user mouth size. In one embodiment, the size of the ring portion may be adjustable if the ring portion comprises two extended prongs (16) which do or do not engage each other. If the two ends (18) do interact, adjustable sizing may be facilitated by any means such as complementary teeth (20) or pin and hole (not shown) arrangements which allow repositioning and reengagement of the two ends.

The tongue depressant portion (14) is centrally positioned so as to engage the base (B) of the user's tongue, as is shown in FIG. 1. In one embodiment, the tongue depressant portion (14) is a bulbous attachment. In alternative embodiments, the tongue depressant portion (14) may comprise an inflexible portion which is bent to engage the tongue in the desired manner. The tongue depressant section is positioned and sized to provide relatively gentle pressure to the base of the tongue in the forward direction. It may also provide some gentle pressure downwardly. This forward and/or downward pressure opens the airway of the user, and assists in maintaining the open airway.

In one embodiment, one or more portions of the appliance may be made to comprise a fluorescent material, which would allow a user to locate and pick up the appliance at night without turning on a light. Fluorescent materials are well known and commercially available.

As will be apparent to those skilled in the art, various modifications, adaptations and variations of the foregoing specific disclosure can be made without departing from the scope of the invention claimed herein.

What is claimed is:

1. An oral appliance comprising:
(a) a flexible, mouldable ring portion, adapted to fit between a user's lower teeth and lower lip, or a user's upper teeth and upper lip; and
(b) a tongue depressant portion, adapted to engage the base of a user's tongue.

2. The oral appliance of claim 1 wherein the ring portion comprises a metal wire encased in plastic.

3. The oral appliance of claim 1 where the ring portion comprises a closed loop.

4. The oral appliance of claim 1 wherein the ring portion comprises two extended prongs.

5. The oral appliance of claim 4 wherein the ring portion extended prongs releasably engage each other at a distal end of each prong.

6. A method of maintaining an airway during sleep, comprising the step of maintaining pressure on the base of a user's tongue during sleep, to push the tongue outward or downward, or both outward and downward, with an oral appliance.

7. The method of claim 6 wherein the oral appliance comprises a flexible, mouldable ring portion, adapted to fit between a user's lower teeth and lower lip, or a user's upper teeth and upper lip, and a tongue depressant portion which engages the base of the user's tongue.

8. The method of claim 6 which is a method of reducing or preventing snoring.

9. The method of claim 6 which is a method of alleviating the symptoms of sleep apnea.

10. Use of an oral appliance to maintain an airway during sleep, wherein said oral appliance maintains pressure on the base of a user's tongue.
